(19) United States
(12) Patent Application Publication
(10) Pub. No.: US 2020/0330634 A1
(43) Pub. Date: Oct. 22, 2020

(54) METHOD FOR IMPROVED FLOW WITH OSCILLATION FOR STERILIZATION OF DEVICES

(71) Applicant: Medivators Inc., Minneapolis, MN (US)

(72) Inventors: Judson A. Herrig, Elko New Market, MN (US); Maruti Sinha, Plymouth, MN (US)

(21) Appl. No.: 16/850,404

(22) Filed: Apr. 16, 2020

Related U.S. Application Data
(60) Provisional application No. 62/836,911, filed on Apr. 22, 2019.

(51) Int. Cl. A61L 2/24; A61L 2/26; A61L 2/07

(52) U.S. Cl. CPC A61L 2/24; A61L 2/26; A61L 2/07; A61L 2202/18; A61L 2202/24; A61L 2202/15; A61L 2202/122

(57) ABSTRACT

A decontamination system for a device, such as a lumen device is provided. The decontamination system includes a terminal package dimensioned to receive a device for decontamination. A decontamination chamber is provided that is dimensioned to receive the terminal package. The system includes a sterilant fluid delivery device configured to deliver a sterilant fluid to the decontamination chamber. A pressure pulse generator is included that is configured to generate flow oscillations in one or more of the terminal package or the decontamination chamber.

### PRIORITY CLAIM

This application claims priority to and benefit of U.S. Provisional Application with Ser. No. 62/836,911 filed Apr. 22, 2019, entitled METHOD FOR IMPROVED FLOW WITH OSCILLATION IN ENDOSCOPE LUMENS, which is herein incorporated by reference in its entirety.

### TECHNICAL FIELD

This disclosure relates generally to decontamination of medical devices; in particular, this disclosure relates to a pressure pulse generator for applying flow oscillations in a decontamination system.

### BACKGROUND

Robust medical instruments are often sterilized at high temperatures. Commonly, the instruments are sterilized in a steam autoclave under a combination of high temperature and pressure. While such sterilization methods are very effective for more durable medical instruments, advanced medical instruments formed of rubber and plastic components with adhesives are delicate and wholly unsuited to the high temperatures and pressures associated with a conventional steam autoclave. Steam autoclaves have also been modified to operate under low pressure cycling programs to increase the rate of steam penetration into the medical devices or associated packages of medical devices undergoing sterilization. Steam sterilization using gravity, high pressure or pre-vacuum create an environment where rapid changes in temperature can take place. In particular, highly complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

Endoscopes can also present problems in that such devices typically have numerous exterior crevices and interior lumens which can harbor microbes. Microbes can be found on surfaces in such crevices and interior lumens as well as on exterior surfaces of the endoscope. Other medical or dental instruments which comprise lumens, crevices, and the like can also provide challenges for decontaminating various internal and external surfaces that can harbor microbes.

Therefore, a need exists that overcomes one or more of the disadvantages of present decontamination systems.

### SUMMARY OF THE INVENTION

According to one aspect, this disclosure provides a decontamination system for a device, such as a lumen device. The decontamination system includes a terminal package dimensioned to receive a device for decontamination. A decontamination chamber is provided that is dimensioned to receive the terminal package. The system includes a sterilant fluid delivery device configured to deliver a sterilant fluid to the decontamination chamber. A pressure pulse generator is included that is configured to generate flow oscillations in one or more of the terminal package or the decontamination chamber.

According to another aspect, this disclosure provides a pressure pulse generator for a decontamination system. The pressure pulse generator includes a diaphragm and a piston. The diaphragm is movable between a first position and a second position to generate pressure pulses.

The piston configured to oscillate. In some cases, the diaphragm moves between the first position and the second position responsive to oscillation of the piston.

According to a further aspect, this disclosure provides a method of decontaminating a device, such as a lumen device. The method includes the step of providing a decontamination chamber of a decontamination system. A sterilant fluid is delivered to the decontamination chamber. Next, a plurality of flow oscillations are applied to an interior of the decontamination chamber to agitate flow within the decontamination chamber.

### BRIEF DESCRIPTION OF THE DRAWINGS

The present disclosure will be described hereafter with reference to the attached drawings which are given as non-limiting examples only, in which:

FIG. 1 is diagrammatic view of a system for decontaminating a medical device according to an embodiment of the present disclosure;

FIG. 2 is a side cross-sectional view of an example pressure pulse generator according to a first embodiment of the present disclosure;

FIG. 3 is a side diagrammatical view of an example pressure pulse generator according to a second embodiment of the present disclosure;

FIG. 4 is a diagrammatical view of a decontamination system with a pressure pulse generator according to a first embodiment of the present disclosure;

FIG. 5 is a diagrammatical view of a decontamination system with a pressure pulse generator according to a second embodiment of the present disclosure;

FIG. 6 is a diagrammatical view of a decontamination system with a pressure pulse generator according to a third embodiment of the present disclosure; a

FIG. 7 is a diagrammatical view of a decontamination system with a pressure pulse generator according to a fourth embodiment of the present disclosure.

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

This disclosure relates to a decontamination system for decontaminating a device, such as a lumen device. In some embodiments, the system includes a pressure pulse generator that generates flow oscillations during decontamination cycles, which aids in forcing sterilant fluid into a device. These oscillations increase flow of sterilant fluid in the device, which may result in better exposure of the device to the sterilant fluid. Depending on the circumstances, the oscillations could be produced by a vibration device, such as a reciprocating piston pump or diaphragm pump driven by an electromagnetic coil. In some cases, harmonics could be used to drive sterilant fluid into or out of the device.

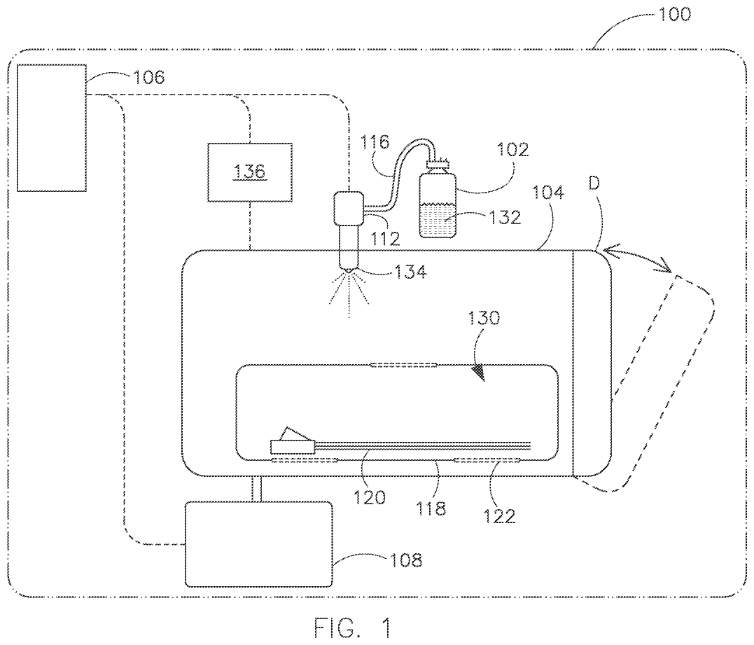

FIG. 1 is a diagrammatic view of one embodiment of a system 100 for decontaminating a medical, dental, or other device having one or more lumens extending there-through. The system 100 includes a reservoir 102, a decontamination chamber 104, a system controller 106, an environmental monitoring and control system 108, and vaporizer 112 which is connected to the reservoir 102 by conduit 116. A terminal package 118 containing a device 120 for decontamination may be placed within the decontamination chamber 104. In the illustrated embodiment, the terminal package 118 includes a fluid inlet, which could be in the form of a plurality of openings or pores 122. The reservoir 102 may be in fluid communication with the decontamination chamber 104 via vaporizer 112. This provides sterilant fluid to the interior of the decontamination chamber 104 for decontaminating the device 120. Although FIG. 1 shows vaporized delivery of sterilant fluid via vaporizer 112 for purposes of example, the sterilant fluid could be introduced via one or more fluid conduits coupled to port(s) of the device 120, or in other ways depending on the circumstances.

The system controller 106 provides control signals to and/or receives condition sensing and equipment status signals from the reservoir 102, the decontamination chamber 104, environmental monitoring and control system 108, and/or the vaporizer 112. In some embodiments, the system 100 can be assembled in a device small enough to sit on a tabletop or counter. For example, the decontamination chamber 104 may have an interior volume of less than about ten cubic feet.

The device 120 to be decontaminated can be placed into the decontamination chamber 104 by opening the door D and placing the device 120 on a rack or other supporting assembly in the interior of the decontamination chamber 104. In some embodiments, the device 120 may be enclosed in the terminal package 118 before being placed in the decontamination chamber 104. In the example shown, the terminal package 118 defines a device receiving area, such as a lumen device receiving area, 130 to receive the device 120 for decontamination. In the illustrated embodiment, the terminal package 118 includes a plurality of openings or pores 122.

The reservoir 102 may be a holding tank or other assembly configured to hold a sterilant fluid 132. In some embodiments, the sterilant fluid 132 can be a chemical or other substance suitable for use in a sterilization process that complies with the International Organization for Standardization (ISO) standard ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005). In some embodiments, the sterilant fluid 132 can be a room temperature (e.g., 20° C. to 25° C.) substance that can be dispersed as a fluid, such as a liquid, a vapor, or a combination thereof (such as a fog) during the decontamination process. Suitable substances for the sterilant fluid 132 include hydrogen peroxide (H₂O₂) and peracetic acid (PAA).

In various embodiments, the sterilant fluid is a composition that includes: (a) hydrogen peroxide; (b) organic acid; (c) a polymeric sulfonic acid resin based chelator; and (d) surfactant. The composition includes less than about 1 wt. % of an anticorrosive agent. The composition can further optionally include water.

In one aspect, the hydrogen peroxide present in the composition can be from about 0.5 wt. % to about 30 wt. %, from about 0.5 wt. % to about 1.5 wt. %, from about 0.8 wt. % to about 1.2 wt. %, from about 20 wt. % to about 30 wt. % and all ranges and values from about 0.5 wt. % to about 30 wt. %.

In another aspect, the acetic acid present in the composition can be from about 1 wt. % to about 25 wt. %, from about 4 wt. % to about 20 wt. %, from about 4.5 wt. % to about 5.5 wt. %, from about 9 wt. % to about 17 wt. % and all ranges and values from about 1 wt. % to about 25 wt. %.

In still another aspect, the peracetic acid present in the composition can be from about 0.01 wt. % to about 25 wt. %, from about 0.05 wt. % to about 20 wt. %, from about 0.05 wt. % to about 0.1 wt. %, from about 3.5 wt. % to about 8 wt. % and all ranges and values from about 0.01 wt. % to about 25 wt. %.

In yet another aspect, the polymeric resin chelator present in the composition can be from about 0.1 wt. % to about 5 wt. %, from about 0.2 wt. % to about 2 wt. %, from about 0.5 wt. % to about 1.5 wt. % and all ranges and value from about 0.1 wt. % to about 5 wt. %.

In various embodiments, the present invention provides for a composition that includes: (a) hydrogen peroxide, present in a concentration of about 0.5 wt. %to about 30 wt. %, e.g., about 28 wt. %; (b) acetic acid, present in a concentration of about 3 wt. % to about 25 wt. %, e.g., about 16 wt. %; (c) a sulfonic acid supported polymeric resin chelator present in a concentration of about 0.1 wt. % to about 5 wt. %, e.g., about 0.2 wt. % to about 0.7 wt. %; and, optionally, (d) Pluronic® 10R5 surfactant block copolymer, present in a concentration of about 2.0 wt. %, wherein the composition comprises less than about 0.1 wt. % of an anticorrosive agent, e.g., 0 wt. % of an anticorrosive agent. The composition can further optionally include water. In some embodiments, the hydrogen peroxide and acetic acid can combine to form peracetic acid, present in about 4 wt. % to about 8 wt. %, e.g., 6.8-7.5 wt. %.

In certain aspects, the peracetic acid/hydrogen peroxide compositions are stabilized without the need for a phosphonic based chelator, such as 1-hydroxyethylidene-1,1,-diphosphonic acid. In other aspects, a phosphonic based chelator, such as 1-hydroxyethylidene-1,1,-diphosphonic acid can be included in the sterilant fluid and therefore, component c), the polymeric sulfonic acid resin is optional. This is detailed in pending PCT application PCT/US19/53090, filed Sep. 26, 2019, entitled “Peracetic Acid Stabilized Compoistions with Polymeric Resins Chelators”, the contents of which are incorporated herein by reference.

The terminal package 118 is sized so that the device 120 to be decontaminated fits within the terminal package 118. In some embodiments, the terminal package 118 may be generally described as having a top, a bottom, and four sides extending between the top and bottom to create a cube-like structure. However, the terminal package 118 may have any suitable shape which encloses the device 120. In some embodiments, the terminal package 118 may be formed from a rigid material such that the terminal package 118 has a rigid or structured shape. Alternatively, the terminal package 118 may be formed from a flexible material such that the terminal package 118 has a flexible shape. Suitable materials for the terminal package 118 include but are not limited to a polymeric non-woven sheet, such as spun-bonded polyethylene (e.g., Tyvek®, sold by E.I. du Pont de Nemours and Company, Wilmington, Del.), and polymeric materials such as polyester and polypropylene. Suitable materials for terminal package 118 having a rigid or structured shape include but are not limited to various metals such as aluminum, stainless steel and/or various polymers in rigid form such as polyethylene and/or polypropylene.

The device 120 may be positioned within the terminal package 118 and subjected to one or more decontamination cycles. Suitable devices include any medical, dental or other device, such as those having at least one lumen extending through at least a portion of the device. In some embodiments, the device 120 may include at least one lumen extending the entire length of the device. For example, the device 120 may be an endoscope.

The terminal package 118 may be configured to prevent or reduce microbes and/or other contaminants from entering the terminal package 118. In some embodiments, for example, the terminal package 118 can include a material suitable for allowing flow of a sterilant fluid, such as hydrogen peroxide (H₂O₂) and/or peracetic acid (PAA), into the device receiving area 130 of the terminal package 118 and blocking or reducing the flow of contaminants into the interior of the terminal package 118. In the illustrated embodiment, the terminal package 118 includes a plurality of openings or pores 122 for allowing flow of the sterilant fluid 132 into the terminal package 118. In some embodiments, the pores 122 may be sized so as to allow the sterilant fluid 132 and/or air to communicate into and out of the container 118 as well as prevent microbes from entering the terminal package 118.

In some embodiments, the sterilant fluid 132 can flow from the reservoir 102 to vaporizer 112 and subsequently to decontamination chamber 104 and device 120. The amount of sterilant fluid 132 introduced into the decontamination chamber 104, the device 120 or a combination thereof can be controlled by the system controller 106 by controlling the amount of the sterilant fluid 132 fed or delivered to vaporizer 112. The rate and amount of the sterilant fluid 132 delivered to vaporizer 112 may be preprogrammed into the system controller 106 or may be manually entered into the system controller 106 by a user of the system 100.

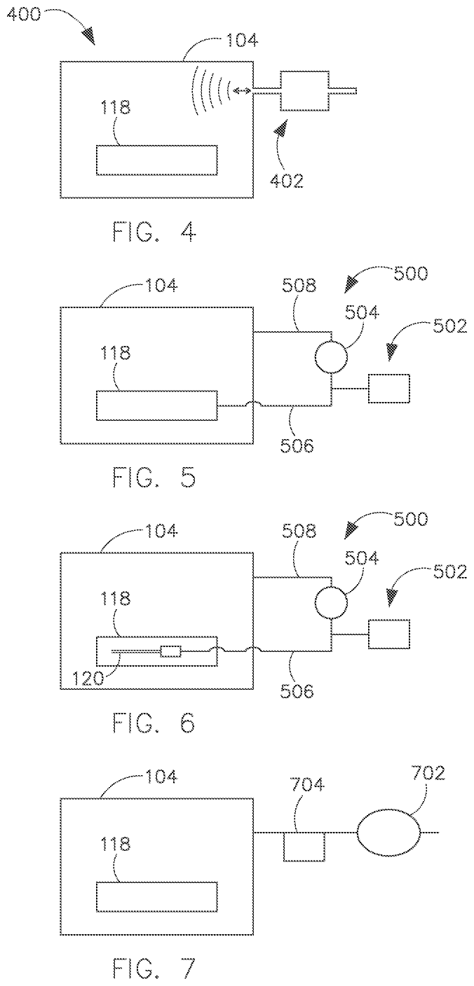

In the embodiment shown, the system 100 includes a pressure pulse generator 136 to generate fluid oscillations within the decontamination chamber 104. The fluid oscillations created by the pressure pulse generator 136 produces movement of the sterilant fluid 132 within the decontamination chamber 104, which tends to increase exposure of the sterilant fluid in the device 120. Although FIG. 1 shows the pressure pulse generator 136 connected to the decontamination chamber 104, the pressure pulse generator 136 could be connected to the terminal package 118, the device 120, vaporizer 112, or other components of the system 100 to introduce flow oscillations. FIGS. 4-7 illustrate example configurations in which a pressure pulse generator could be configured to introduce flow oscillations in conjunction with the system 100 as discussed below.

To decontaminate a device, such as a lumen device, such as a medical, dental or other device, the device 120 may be sealed within the terminal package 118 and placed in the decontamination chamber 104. The device 120 is then subjected to a decontamination process which may include one or more decontamination cycles. A suitable cycle may include adjusting the pressure of the decontamination chamber 104 to a suitable range, such as to a pressure less than 10 Torr, conditioning using plasma, and introducing the sterilant fluid 132 into the decontamination chamber 104 via vaporizer 112 and nozzle 134. The sterilant fluid 132 may be held within the decontamination chamber 104 for a period of time to facilitate the decontamination of the device 120, and in particular, the exterior surfaces of the device 120. Similarly, the sterilant fluid 132 may be held within the device 120 for a period of time to facilitate the decontamination of any interior surfaces or lumen(s) of the device 120. When the sterilant fluid 132 has been held in the decontamination chamber 104 for the desired or programmed amount of time, the system controller 106 can vent the decontamination chamber 104 to a higher, but sub-atmospheric pressure. The system controller 106 can then hold the pressure within the decontamination chamber 104 for a period of time to further facilitate the decontamination of the load. Following the hold period, the system controller 106 may evacuate the decontamination chamber 104 to remove the sterilant fluid residuals from the decontamination chamber 104 which may also include a plasma treatment to further enhance the removal of the substance residuals, followed by venting the decontamination chamber 104. This cycle or steps may be repeated or extended as part of a comprehensive cycle.

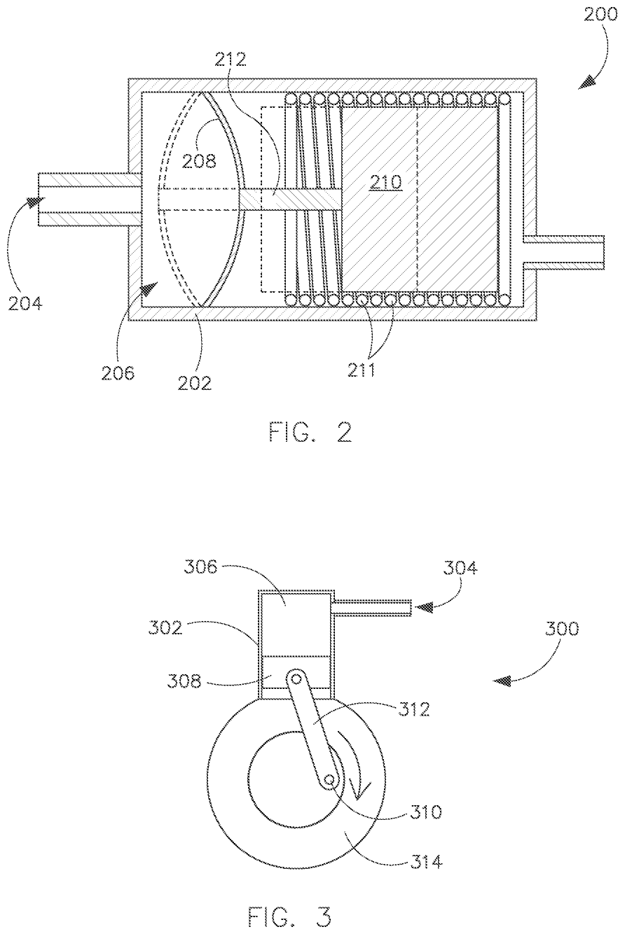

FIG. 2 is a side cross-sectional diagrammatical view of an example pressure pulse generator 200 according to an embodiment of this disclosure. In the embodiment shown, the pressure pulse generator 200 includes a body 202 defining an interior cavity dimensioned to house internal components. As shown, the body 202 includes a port 204 through which pressure pulses exit. Typically, the port 204 is fluidly connected with the interior of the decontamination chamber 104 and/or in fluid communication with the terminal package 118. A charging cavity 206 is defined between the port 204 and a diaphragm 208. The diaphragm 208 is movable between a first position (solid line) and a second position (dashed line). When the diaphragm 208 moves from the first position to the second position, this ejects fluid, such as air and/or sterilant fluid, within the charging cavity 206 out of the port 204. When the diaphragm 208 moves from the second position to the first position, this tends to draw fluid, such as air and/or sterilant fluid, through the port 204 into the charging cavity 206. As such, this generates flow oscillations into/out of the port 204.

In the embodiment shown, the diaphragm moves between the first position and the second position responsive to oscillations of a piston 210. As shown, the piston 210 oscillates between a first position (solid line) and a second position (dashed line). For example, the piston 210 could be formed, at least in part, from a ferrous material. A wire coil 211 surrounds the piston 210 to generate a magnetic field. For example, the wire coil 211 could be electrically connected to an input signal to selectively control the strength of the magnetic field that is generated. In some embodiments, there could be a magnet disposed in or on the body 202. The interaction between the magnetic field generated by the wire coil 212 and the magnet in/on the body 202 causes the piston 210 to oscillate. In the embodiment shown, a rod 212 extends from the piston 210 to engage the diaphragm 208. As shown, the rod 212 moves the diaphragm 208 between the first and second positions as the piston 210 oscillates. In the example shown, the body 202 defines a vent port 214 to vent fluid (e.g., air) out of the body 202 behind the piston 210 as the piston 210 oscillates.

FIG. 3 is a side diagrammatical view of an example pressure pulse generator 300 according to another embodiment of this disclosure. In this embodiment, the pressure pulse generator 300 includes a body 302 defining an interior cavity dimensioned to house internal components. As shown, the body 302 includes a port 304 through which pressure pulses exit. Typically, the port 304 is fluidly connected with the interior of the decontamination chamber 104 and/or in fluid communication with the terminal package 118. A charging cavity 306 is defined between the port 304 and a piston 308. The piston 308 is movable between a first position and a second position. When the piston 308 moves between the first position and the second position, fluid, such as air and/or sterilant fluid, within the charging cavity 306 is ejected out and drawn in through the port 304. As such, this generates flow oscillations into/out of the port 304 as the piston 308 oscillates.

In the example shown, the piston 308 is pivotally coupled with a crank 310 using a connecting rod 312. As shown, the connecting rod 312 has a first end pivotally coupled with the crank 310 and a second end pivotally coupled with the piston 308. In this example, the crank 310 is pivotally coupled with a rotating body 314. This translates the rotation of the rotating body 314 into a linear oscillation of the piston 308, which generates pressure pulses out the port 304.

FIGS. 4-7 illustrate example configurations of a pressure pulse generator in a decontamination system to enhance sterilant fluid 132 exposure to the device 120. FIG. 4 is a diagrammatic view of a decontamination system 400 with an example pressure pulse generator 402 according to an embodiment. As shown, the pressure pulse generator 402 is in fluid communication with the interior of the decontamination chamber 104. The pressure pulse generator 402 is configured to create flow oscillations within the decontamination chamber 104. Although this example shows a pressure pulse generator 402 similar to the embodiment of pressure pulse generator 200 shown in FIG. 2, the pressure pulse generator 300 shown in FIG. 3 could instead be used.

FIG. 5 is a diagrammatic view of a decontamination system 500 with an example pressure pulse generator 502 according to another embodiment. In the embodiment shown, a recirculating pump 504 has an outlet 506 that delivers sterilant fluid to the terminal package 118. The reciprocating pump 504 has an inlet 508 connected with the reciprocating pump 504 to recirculate sterilant fluid from within the decontamination chamber 104 back to the outlet 506 of the reciprocating pump 504. As shown, the pressure pulse generator 502 applies fluid oscillations to the outlet 506 of the pump 504. These oscillations may increase exposure of sterilant fluid within the device. FIG. 6 illustrates a similar configuration to that shown in FIG. 5, but with the outlet 506 of the pump 504 directly connected with one or more ports on the device 120.

FIG. 7 is a diagrammatic view of a decontamination system 700 according to another embodiment. In the embodiment shown, the pressure pulse generator 502 includes a vacuum pump 702 in fluid communication with the decontamination chamber 104. An electronic valve 704 selectively controlling fluid communication between the vacuum pump 702 and the interior of the decontamination chamber 104. For example, the electronic valve 704 may be controlled with an input signal to open and close the valve. This can be used to vary the pressure with which the vacuum pump 702 draws fluid out of the decontamination chamber 104.

### EXAMPLES

Illustrative examples of the method and system disclosed herein are provided below. An embodiment of the method and system may include any one or more, and any combination of, the examples described below.

Example 1 is a decontamination system for a device, such as a lumen device. The decontamination system includes a terminal package dimensioned to receive a device for decontamination. A decontamination chamber is provided that is dimensioned to receive the terminal package. The system includes a sterilant fluid delivery device configured to deliver a sterilant fluid to the decontamination chamber. A pressure pulse generator is included that is configured to generate flow oscillations in one or more of the terminal package or the decontamination chamber.

In Example 2, the subject matter of Example 1 is further configured such that the pressure pulse generator is configured to fluctuate pressure output of the sterilant fluid delivery device.

In Example 3, the subject matter of Example 1 is further configured such that the decontamination chamber defines a port into which the pressure pulse generator injects flow oscillations.

In Example 4, the subject matter of Example 1 is further configured such that the pressure pulse generator is in fluid communication with the terminal package.

In Example 5, the subject matter of Example 4 is further configured such that the pressure pulse generator is in fluid communication with the sterilant fluid delivery device.

In Example 6, the subject matter of Example 5 is further configured such that the pressure pulse generator is configured to fluctuate pressure of sterilant fluid delivered to the terminal package.

In Example 7, the subject matter of Example 5 is further configured such that the sterilant fluid delivery device comprises a reciprocating pump.

In Example 8, the subject matter of Example 7 is further configured such that the pressure pulse generator is fluidly inline between an outlet of the reciprocating pump and the terminal package.

In Example 9, the subject matter of Example 4 is further configured such that the pressure pulse generator includes a vacuum pump in fluid communication with the terminal package, wherein the vacuum pump is configured to draw fluid from the device in the terminal package.

In Example 10, the subject matter of Example 9 is further configured such that the pressure pulse generator includes an electrically-controlled valve configured to selectively control pressure with which fluid is drawn from the vacuum pump.

In Example 11, the subject matter of Example 1 is further configured such that the pressure pulse generator includes a diaphragm movable between a first position and a second position to generate pressure pulses.

In Example 12, the subject matter of Example 11 is further configured such that the pressure pulse generator includes a piston configured to oscillate, and wherein the diaphragm moves between the first position and the second position responsive to oscillation of the piston.

In Example 13, the subject matter of Example 1 is further configured such that the piston includes an electro-magnet portion that oscillates responsive to changes in an electrical input frequency.

In Example 14, the subject matter of Example 12 is further configured such that the piston oscillates responsive to rotational movement of a crank.

Example 15 is a pressure pulse generator for a decontamination system. The pressure pulse generator includes a diaphragm and a piston. The diaphragm is movable between a first position and a second position to generate pressure pulses. The piston configured to oscillate. In some cases, the diaphragm moves between the first position and the second position responsive to oscillation of the piston.

In Example 16, the subject matter of Example 15 is further configured such that the piston includes an electro-magnet portion that oscillates responsive to changes in an electrical input frequency.

In Example 17, the subject matter of Example 15 is further configured such that the piston oscillates responsive to rotational movement of a crank.

In Example 18, the subject matter of Example 15 is further configured to include a rod extending from the piston that is configured to move concomitant with oscillation of the piston.

In Example 19, the subject matter of Example 18 is further configured such that the rod is configured to engage the diaphragm to move between the first position and the second position as the piston oscillates.

Example 20 is a method of decontaminating a device, such as a lumen device. The method includes the step of providing a decontamination chamber of a decontamination system. A sterilant fluid is delivered to the decontamination chamber. Next, a plurality of flow oscillations are applied to an interior of the decontamination chamber to agitate flow within the decontamination chamber.

Although the present disclosure has been described with reference to particular means, materials and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A decontamination system for a device, the decontamination system comprising:
a terminal package dimensioned to receive a device for decontamination;
a decontamination chamber dimensioned to receive the terminal package;
a sterilant fluid delivery device configured to deliver a sterilant fluid to the decontamination chamber; and
a pressure pulse generator configured to generate flow oscillations in one or more of the terminal package or the decontamination chamber.

2. The decontamination system of claim 1, wherein the pressure pulse generator is configured to fluctuate pressure output of the sterilant fluid delivery device.

3. The decontamination system of claim 1, wherein the decontamination chamber defines a port into which the pressure pulse generator injects flow oscillations.

4. The decontamination system of claim 1, wherein the pressure pulse generator is in fluid communication with the terminal package.

5. The decontamination system of claim 4, wherein the pressure pulse generator is in fluid communication with the sterilant fluid delivery device.

6. The decontamination system of claim 5, wherein the pressure pulse generator is configured to fluctuate pressure of sterilant fluid delivered to the terminal package.

7. The decontamination system of claim 5, wherein the sterilant fluid delivery device comprises a reciprocating pump.

8. The decontamination system of claim 7, wherein the pressure pulse generator is fluidly inline between an outlet of the reciprocating pump and the terminal package.

9. The decontamination system of claim 4, wherein the pressure pulse generator includes a vacuum pump in fluid communication with the terminal package, wherein the vacuum pump is configured to draw fluid from the device in the terminal package.

10. The decontamination system of claim 9, wherein the pressure pulse generator includes an electrically-controlled valve configured to selectively control pressure with which fluid is drawn from the vacuum pump.

11. The decontamination system of claim 1, wherein the pressure pulse generator includes a diaphragm movable between a first position and a second position to generate pressure pulses.

12. The decontamination system of claim 11, wherein the pressure pulse generator includes a piston configured to oscillate, and wherein the diaphragm moves between the first position and the second position responsive to oscillation of the piston.

13. The decontamination system of claim 12, wherein the piston includes an electro-magnet portion that oscillates responsive to changes in an electrical input frequency.

14. The decontamination system of claim 12, wherein the piston oscillates responsive to rotational movement of a crank.

15. A pressure pulse generator for a decontamination system, the pressure pulse generator comprising:
a diaphragm movable between a first position and a second position to generate pressure pulses;
a piston configured to oscillate; and
wherein the diaphragm moves between the first position and the second position responsive to oscillation of the piston.

16. The pressure pulse generator of claim 15, wherein the piston includes an electro-magnet portion that oscillates responsive to changes in an electrical input frequency.

17. The pressure pulse generator of claim 15, wherein the piston oscillates responsive to rotational movement of a crank.

18. The pressure pulse generator of claim 15, further comprising a rod extending from the piston, wherein the rod is configured to move concomitant with oscillation of the piston.

19. The pressure pulse generator of claim 18, wherein the rod is configured to engage the diaphragm to move between the first position and the second position as the piston oscillates.

20. A method of decontaminating a device, the method comprising the steps of:
providing a decontamination chamber of a decontamination system;
delivering a sterilant fluid to the decontamination chamber; and
applying a plurality of flow oscillations to an interior of the decontamination chamber to agitate flow within the decontamination chamber.
